(19) United States
(12) Patent Application Publication
(10) Pub. No.: US 2016/0312220 A1
(43) Pub. Date: Oct. 27, 2016

(54) METHOD AND PHARMACEUTICAL COMPOSITION FOR INHIBITING NEURONAL REMODELING

(71) Applicants: INSERM (Institut NAational de la Santé et de la Recherche Médicale), Paris (FR); CENTRE NATIONAL DE LA RECHERCHE SCIENTIFIQUE (CNRS), Paris (FR); Université d'Aix Marseille, Marseille (FR); Assistance Publique Hôpitaux de Marseille, Marseille (FR); Institut Jean Paoli & Irene Calmettes, Marseille (FR)

(72) Inventors: Richard TOMASINI, Marseille (FR); Juan IOVANNA, Marseille (FR); Véronique SECQ, Marseille (FR)

(21) Appl. No.: 15/102,682

(22) PCT Filed: Dec. 10, 2014
(86) PCT No.: PCT/EP2014/077123

§ 371 Date: Jun. 8, 2016

(30) Foreign Application Priority Data
Dec. 10, 2013 (EP) 13306690.2

(51) Int. Cl. C12N 15/113

(52) U.S. Cl. CPC C12N 15/113; C12N 2310/14; C12N 2320/30

(57) ABSTRACT

The present disclosure relates to a compound which inhibits the binding of SLIT2 to ROBO or ROB02 or a compound which is an inhibitor of SLIT2, ROBO1 or ROB02 gene expression for use as an inhibitor of the neuronal remodeling in cancer.

### FIELD OF THE INVENTION

The present invention relates to a compound which inhibits the binding of SLIT2 to ROBO1 or ROBO2 or a compound which is an inhibitor of SLIT2, ROBO1 or ROBO2 gene expression for use as an inhibitor of the neuronal remodeling in cancer.

### BACKGROUND OF THE INVENTION

Even after drastic efforts from scientific community in the last decade, Pancreatic Ductal Adenocarcinoma (PDA) stays one of the most lethal cancers. Regarding the 2013 cancer mortality predictions, it was reported that given the declines in mortality in most major other cancers, pancreatic cancer remains with unfavorable trends and has become the fourth cause of cancer death in both sexes. Median survival stagnates around 5 months together with a 5-years-survival at 5%. Importantly, only 5%-20% of patients present a resectable pancreatic cancer, but even in patients with R0 (healthy margins) resected tumors, 5-years survival is no more than 20% with a median survival between 12 and 20 months. Indeed, high prevalence of local tumor recurrence even after potentially curative resection, which leads to diminished survival, is attributed to the residual tumor cells that are undetected during the surgery. A recent study revealed that overall survival of patients with tumor recurrence was 9.3 months versus 26.3 months for patients without early relapse. Interestingly, local recurrence of pancreatic cancer is principally found in the cut end of the remnant pancreas after initial pancreatic resection, and total remnant pancreatectomy is performed when possible as any second line treatment is at present available. The foremost reason for this local tumor recurrence is related with the presence of an important neural remodeling. In light of such dramatic epidemiological data, the need of developing optimal therapeutic strategies, which impact on tumor recurrence and take into account the cellular composition of these tumors is of crucial interest for the next decade.

Tumors are complex tissues in which mutant cancer cells and subverted normal cells are coexisting to form an intricate network of multiple cell types whom dialogue and fine communication is of vital consideration. This is even more accurate for PDA, as presence of an abundant tumor stroma (intra-tumoral microenvironment) is considered as an emerging hallmark [Hanahan D, et al. 2001]. Composed by extracellular matrix (ECM), fibroblasts and activated fibroblasts (myofibroblasts or Stellate cells or CAFs for Cancer Associated Fibroblasts), blood and lymphatic vessels, nerve fibers and inflammatory cells, this stromal compartment is well defined as particularly active on tumor development, although considered for a long time as a simple support matrix for the development of tumoral epithelial cells. Indeed, numerous clinical and fundamental findings revealed recently that PDA microenvironment drastically influences pancreatic cancer cells behavior as well as resistance to standard chemotherapy and clinical outcome [Mahadevan D, et al. 2007]. Whatever, if genetic changes of tumor epithelial cells have been deeply investigated in the last decades, the role of stromal cells has largely been lagged behind and is nowadays of major interest.

Beyond the presence of an important stromal compartment, another characteristic of PDA is the presence of a modified innervation histologically characterized by an alteration of intrapancreatic nerves, in n early all patients. This includes increased neural density and hypertrophy, pancreatic neuritis as well as intrapancreatic and extrapancreatic perineural invasion (PNI) by cancer cells, and nerve ultra-structure modifications [Ceyhan G O, et al. 2009]. This neural remodeling or PDA associated neural remodeling (PANR) is clinically correlated with neuropathic pain [Lindsay T H, et al. 2005], locoregional spread [Kayahara M, et al. 2007] and is a marker of poor prognosis [Ozaki H, et al. 1999]. As noticed above, neural remodeling is considered as the foremost reason for local tumor recurrence after curative resection, with residual tumor cells present in the remnant pancreas nerves, extrapancreatic nerve plexus, as well as celiac and superior mesenteric ganglia [Hibi T, et al. 2009]. Recently, histologic analysis of nerve plexus invasion in invasive ductal carcinoma of the pancreas and its correlation with prognosis have clearly shown that PNI is an independent prognostic factor, a significant cause of positive resection margins [Deshmukh S D, et al. 2010] and a predictor for recurrence [Shimada K, et al. 2011]. Beyond a clear clinical significance, PANR pathogenesis and associated molecular mechanisms were completely unknown until very recently. Nevertheless, since few years, several studies reported molecules implicated in such phenomenon. Neurotropins such as Artemin, a member of the glial-cell-derived neurotrophic factor (GDNF), and the nerve growth factor (NGF), as well as its receptor p75 (NGFR), or chemokines through the relation CX3CR1-CX3CL1 but also hematopoietic colony stimulating factors (G-CSF and GM-CSF) and their receptors (G-CSFR and GM-CSFRa) have been correlated to PANR. Moreover, other molecules such as Pigment epithelium-derived factor (PEDF), Myelin-associated glycoprotein (MAG, Siglec-4a), the invariant chain of HLA Class II (CD74), the serine protease tissue plasminogen activators, the g-synuclein and pleiotrophin and, more recently the ECM protein Syndecan-2 (SDC-2) were characterized as molecular determinants of PANR. However, even with the help of those findings as well as others, our knowledge on the biology of tumor cell interaction with nerves as well as the impact of nerve modification on PDA progression and patient outcome still remains very poor. Improvement of the understanding of molecular pathways underlying PDA associated neural remodeling and its clinical significance may lead to better prognostic indicators and to innovative therapeutic strategies directed to malignancy, through targeting local recurrence and loco-regional spread as well as cancer-associated pain.

### SUMMARY OF THE INVENTION

In the present study, the inventors conducted microarray transcriptomic analysis of stromal versus tumoral cell compartments from several PDA patients and highlighted a family of “stromal” neurogenic factors impacting on neuroplastic changes associated to PDA. They further examined the specific involvement of the axon guidance molecules pathway “Slit2/Robo” and demonstrated the fundamental role of intra-tumoral microenvironment on PDA associated neural remodeling, through secretion of specific proteins that modulate nerve cells abilities. Their results strongly suggest that inhibiting tumor-stroma interactions, and more specifically the Slit2/Robo axis, could be a promising therapeutic strategy for PDA to hold down processes involved in disease recurrence, loco-regional spread and associated neuropathic pain.

Thus, the invention relates to a compound which inhibits the binding of SLIT2 to ROBO1 or ROBO2 or a compound which is an inhibitor of SLIT2, ROBO1 or ROBO2 gene expression for use as an inhibitor of the neuronal remodeling in cancer.

The invention also relates to a therapeutic composition comprising a compound according to the invention for use as an inhibitor of the neuronal remodeling in cancer.

### DETAILED DESCRIPTION OF THE INVENTION

A first aspect of the invention relates to a compound which inhibits the binding of SLIT2 to ROBO1 or ROBO2 or a compound which is an inhibitor of SLIT2, ROBO1 or ROBO2 gene expression for use as an inhibitor of the neuronal remodeling in cancer.

The invention also relates to i) compound according to the invention, and ii) a chemotherapeutic agent, as a combined preparation for simultaneous, separate or sequential use as an inhibitor of the neuronal remodeling in cancer.

As used herein, the term “neuronal remodeling in cancer” denotes the formation of a new nervous system within the tumoral mass. More specifically, the term “neuronal remodeling in pancreatic cancer” denotes three different physiological changes: 1/an increased neural density with hypertrophic nerves, together with an important neurite outgrowth, 2/an intrapancreatic and extrapancreatic perineural invasion, characterized by an infiltration of those new nerve fibers by cancer cells (promoting cell evasion, metastasis and local recurrence) and immune cells (promoting neuropathic pain), 3/ultra-structure modifications of nerve with changes in signal transmission and activation/differentiation status of nerve cells.

Neuronal remodeling is one of the causes of neuropathic pain and is, at present, the main reason of local recurrence after curative resection of PDAC tumor. Thus, the inhibition of neuronal remodeling, is a putative target for the treatment of neuropathic pain but more importantly cancer progression.

The invention also relates to a compound which inhibits the binding of SLIT2 to ROBO1 or ROBO2 or a compound which is an inhibitor of SLIT2, ROBO1 or ROBO2 gene expression for decrease or relieve the pain in a patient suffering from pancreatic cancer.

Thus, the invention also relates to a compound which inhibits the binding of SLIT2 to ROBO1 or ROBO2 or a compound which is an inhibitor of SLIT2, ROBO1 or ROBO2 gene expression for use in the treatment of neuropathic pain in cancer.

The invention also relates to a compound which inhibits the binding of SLIT2 to ROBO1 or ROBO2 or a compound which is an inhibitor of SLIT2, ROBO1 or ROBO2 gene expression for use in the prevention of cancer recurrence.

The invention also relates to a compound which inhibits the binding of SLIT2 to ROBO1 or ROBO2 or a compound which is an inhibitor of SLIT2, ROBO1 or ROBO2 gene expression for use in the prevention of metastasis.

The invention also relates to a compound which inhibits the binding of SLIT2 to ROBO1 or ROBO2 or a compound which is an inhibitor of SLIT2, ROBO1 or ROBO2 gene expression for slow down the progression of a cancer.

The invention also relates to a compound which inhibits the binding of SLIT2 to ROBO1 or ROBO2 or a compound which is an inhibitor of SLIT2, ROBO1 or ROBO2 gene expression for use as an inhibitor of the neuronal remodeling in cancer to inhibit and prevent side effects in cancer and especially to inhibit and prevent pain, metastasis and cancer recurrence.

The invention also relates to i) compound according to the invention, and ii) a chemotherapeutic agent, as a combined preparation for simultaneous, separate or sequential use in the treatment of neuropathic pain in cancer.

The invention also relates to i) compound according to the invention, and ii) a chemotherapeutic agent, as a combined preparation for simultaneous, separate or sequential use in the prevention of cancer recurrence.

The invention also relates to i) compound according to the invention, and ii) a chemotherapeutic agent, as a combined preparation for simultaneous, separate or sequential use in the prevention of metastasis.

According to the invention chemotherapeutic agent may be Gemcitabine, Paclitaxel, Nab-Placlitaxel, Folfirinox, (folinic acid, 5-fluorouracil, irinotecan, oxaliplatin) or Abraxane.

As used herein, the term “SLIT2” has its general meaning in the art and refers to a gene that encodes for a secreted molecule mainly involved in axon guidance. An exemplary sequence for human SLIT2 protein is deposited in the Uniprot database under accession numbers O94813.

As used herein, the term “ROBO1” for “Roundabout homolog 1” has its general meaning in the art and refers to a member of the immunoglobulin gene superfamily which is an axon guidance receptor and a cell adhesion receptor. An exemplary sequence for human ROBO1 protein is deposited in the UniProt database under accession numbers Q9Y6N7.

As used herein, the term “ROBO2” for “Roundabout homolog 2” has its general meaning in the art and refers to a member of the immunoglobulin gene superfamily which is an axon guidance receptor and a cell adhesion receptor. An exemplary sequence for human ROBO2 protein is deposited in the Uniprot database under accession numbers Q9HCK4.

In one embodiment, the cancer according to the invention is a pancreatic cancer, a prostate cancer or a breast cancer.

In a particular embodiment, the compound according to the invention is used as an inhibitor of the neuronal remodeling in pancreatic cancer.

In another particular embodiment, the compound according to the invention is used as an inhibitor of the neuronal remodeling in pancreatic cancer to inhibit and prevent side effects in pancreatic cancer and especially to inhibit and prevent pain, metastasis and cancer recurrence.

In one embodiment, the pancreatic cancer may be a pancreatic ductal adenocarcinoma or a pancreatic neuroendocrine tumor.

In a particular embodiment, the compound according to the invention is used as an inhibitor of the neuronal remodeling in a pancreatic ductal adenocarcinoma to inhibit and prevent side effects in neuroendocrin pancreatic cancer and especially to prevent cancer recurrence.

In a particular embodiment, the compound according to the invention is used as an inhibitor of the neuronal remodeling in a pancreatic neuroendocrin tumor to inhibit and prevent side effects in neuroendocrin pancreatic cancer and especially to prevent cancer recurrence.

In another particular embodiment, the compound according to the invention is used for the treatment of cancer and the treatment of neuropathic pain in cancer in a patient with a resected cancer.

In another particular embodiment, the compound according to the invention is used for the treatment of cancer and the treatment of neuropathic pain in cancer in a patient with a resected pancreatic cancer.

In one embodiment, the compound according to the invention may bind to SLIT2, ROBO1 or ROBO2 and block the binding of SLIT2 on ROBO1 or ROBO2 and block its physiological effects. To identify a compound able to block the interaction between SLIT2, ROBO1 or ROBO2, a test may be used. For example, the compound to test will compete with the binding of SLIT2 labelled with a flurochrom (as fluorescein isothiocyanate) on ROBO1 or ROBO2 transfected cell lines. The inhibition of the binding will be then analyzed by flow cytometry Inhibition of Slit2 signalling and impact on migration/proliferation will be confirmed through already set up assay.

Typically, the compound according to the invention includes but is not limited to a small organic molecule, an antibody, and a polypeptide.

In one embodiment, the compound according to the invention may be a low molecular weight compound, e.g. a small organic molecule (natural or not).

The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

In one embodiment, the compound according to the invention is an antibody. Antibodies directed against SLIT2, ROBO1 or ROBO2 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against SLIT2, ROBO1 or ROBO2 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-SLIT2, anti-ROBO1 or anti-ROBO2 single chain antibodies. Compounds useful in practicing the present invention also include anti-SLIT2, anti-ROBO1 or anti-ROBO2 antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to SLIT2, ROBO1 or ROBO2.

Humanized anti-SLIT2, anti-ROBO1 or anti-ROBO2 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of SLIT2, ROBO1 or ROBO2 are selected.

In one embodiment, the compound according to the invention is an anti-SLIT2 antibody.

In a particular embodiment, the antibody according to the invention may be the ab7665 antibody, the ab82131 antibody or the ab134166 antibody bought by Abcam or the sc-28945 antibody bought by Santa Cruz.

In another embodiment, the compound according to the invention is an anti-ROBO1 antibody.

In a particular embodiment, the antibody according to the invention may be an antibody according to the patent application NZ601733.

In a particular embodiment, the antibody according to the invention may be an antibody according to the patent application US2009092544.

In a particular embodiment, the antibody according to the invention may be an antibody according to the patent application US2007212359.

In a particular embodiment, the antibody according to the invention may be the ab7279 antibody or the ab58297 antibody bought by Abcam.

In a particular embodiment, the antibody according to the invention may be monoclonal antibody R5 according to Wang L J, et al. 2008

In another embodiment, the compound according to the invention is an anti-ROBO2 antibody.

In a particular embodiment, the antibody according to the invention may be the ab64158 antibody or the ab75014 antibody bought by Abcam.

In a particular embodiment, the antibody according to the invention may be a monoclonal antibody R5 according to Hivert B, et al. 2002.

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of SLIT2, ROBO1 or ROBO2 are selected.

In one embodiment, the compound according to the invention is a polypeptide.

In a particular embodiment the polypeptide is a functional equivalent of SLIT2, ROBO1 or ROBO2. As used herein, a “functional equivalent” of SLIT2, ROBO1 or ROBO2 is a compound which is capable of binding to SLIT2, thereby preventing its interaction with ROBO1 or ROBO2. The term “functional equivalent” includes fragments, mutants, and muteins of SLIT2, ROBO1 or ROBO2. The term “functionally equivalent” thus includes any equivalent of ROBO1 or ROBO2 obtained by altering the amino acid sequence, for example by one or more amino acid deletions, substitutions or additions such that the protein analogue retains the ability to bind to SLIT2. Amino acid substitutions may be made, for example, by point mutation of the DNA encoding the amino acid sequence.

Functional equivalents include molecules that bind SLIT2 and comprise all or a portion of the extracellular domains of ROBO1 or ROBO2. Typically, said functional equivalents may be the extracellular domains of ROBO1 or ROBO2 expressed as Fc fusion protein. For example, fusion proteins may be composed of the extracellular ligand binding portion of ROBO1 which blocks activation of ROBO1 by SLIT2 or a fusion protein composed of the extracellular ligand-binding portion of ROBO1 or ROBO2 which blocks activation of ROBO1 or ROBO2 by SLIT2. Such fusion proteins can be generated using methods known in the art, such as recombinant DNA technology as is described in details herein below.

In one embodiment, the polypeptide according to the invention is able to inhibit the neuronal remodeling in cancer through its properties of decoy receptor.

By “decoy receptor”, is meant that the polypeptide according to the invention trap SLIT2 and prevent its physiological effects on ROBO1 or ROBO2.

The functional equivalents include soluble forms of ROBO1 or ROBO2. A suitable soluble form of these proteins, or functional equivalents thereof, might comprise, for example, a truncated form of the protein from which the transmembrane domain has been removed by chemical, proteolytic or recombinant methods.

Preferably, the functional equivalent is at least 80% homologous to the corresponding protein. In a particular embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of ROBO1 or ROBO2 that binds to SLIT2. Accordingly the present invention provides a polypeptide capable of inhibiting binding of ROBO1 or ROBO2 to SLIT2, which polypeptide comprises consecutive amino acids having a sequence which corresponds to the sequence of at least a portion of an extracellular domain of ROBO1 or ROBO2, which portion binds to SLIT2. In one embodiment, the polypeptide corresponds to an extracellular domain of ROBO1 or ROBO2. In another embodiment, the polypeptide corresponds to the extracellular domains of ROBO1 or ROBO2 expressed as Fc fusion protein.

Functionally equivalent fragments may belong to the same protein family as the ROBO1 or ROBO2 identified herein. By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

In one embodiment, the polypeptide according to the invention may be also a functional equivalent of SLIT2. As used herein, a “functional equivalent” of SLIT2 is a compound which is capable of binding to ROBO1 or ROBO2, thereby preventing its interaction with the natural ligand SLIT2. The term “functional equivalent” includes fragments, mutants, and muteins of SLIT2. The term “functionally equivalent” thus includes any equivalent of SLIT2 obtained by altering the amino acid sequence, for example by one or more amino acid deletions, substitutions or additions such that the protein analogue retains the ability to bind to ROBO1 or ROBO2. Amino acid substitutions may be made, for example, by point mutation of the DNA encoding the amino acid sequence.

The polypeptides of the invention may be produced by any suitable means, as will be apparent to those of skill in the art. In order to produce sufficient amounts of SLIT2, ROBO1 or ROBO2 or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the compound according to the invention is an inhibitor of SLIT2, ROBO1 or ROBO2 gene expression.

Small inhibitory RNAs (siRNAs) can also function as inhibitors of SLIT2, ROBO1 or ROBO2 expression for use in the present invention. SLIT2, ROBO1 or ROBO2 gene expression can be reduced by contacting a subject or cell with a small double stranded RNA (dsRNA), or a vector or construct causing the production of a small double stranded RNA, such that SLIT2, ROBO1 or ROBO2 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In a particular embodiment, the anti-SLIT2 siRNA according to the invention may be the siRNA as described in Dickinson R E, et al. 2011 or the siRNA EHU068081 bought by Sigma-Aldrich.

In a particular embodiment, the anti-ROBO1 siRNA according to the invention may be for example the siRNA as described in Huang L, et al. 2009.

Ribozymes can also function as inhibitors of SLIT2, ROBO1 or ROBO2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of SLIT2, ROBO1 or ROBO2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of SLIT2, ROBO1 or ROBO2 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing SLIT2, ROBO1 or ROBO2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and micro encapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

Another object of the invention relates to a method for inhibiting the neuronal remodeling in cancer comprising administering to a subject in need thereof a therapeutically effective amount of a compound which inhibits the binding of SLIT2 to ROBO1 or ROBO2 or a compound which is an inhibitor of SLIT2, ROBO1 or ROBO2 gene expression.

In another embodiment, the invention relates to a method for treating neuropathic pain in cancer comprising administering to a subject in need thereof a therapeutically effective amount of a compound which inhibits the binding of SLIT2 to ROBO1 or ROBO2 or a compound which is an inhibitor of SLIT2, ROBO1 or ROBO2 gene expression.

In another embodiment, the invention relates to a method for preventing cancer recurrence comprising administering to a subject in need thereof a therapeutically effective amount of a compound which inhibits the binding of SLIT2 to ROBO1 or ROBO2 or a compound which is an inhibitor of SLIT2, ROBO1 or ROBO2 gene expression.

### Therapeutic Composition

Another object of the invention relates to a therapeutic composition comprising a compound according to the invention for use as an inhibitor of the neuronal remodeling in cancer.

In one embodiment, the invention relates to a therapeutic composition comprising a compound according to the invention for use as an inhibitor of the neuronal remodeling in cancer to inhibit and prevent side effects in cancer and especially to inhibit and prevent pain, metastasis and cancer recurrence.

In one embodiment, the invention relates to a therapeutic composition comprising a compound according to the invention for treating neuropathic pain in cancer.

In another embodiment, the invention relates to a therapeutic composition comprising a compound according to the invention for preventing cancer recurrence.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising a compound according to the invention and a further therapeutic active agent.

In one embodiment said therapeutic active agent may be an anti-cancer agent or an analgesic agent like morphine.

The invention will be further illustrated by the following figures and examples. However, these examples and figures should not be interpreted in any way as limiting the scope of the present invention.

### FIGURES

FIG. 1. SLIT2/ROBO pathway impacts on neural cells behaviors linked to PDA associated neural remodeling. (A-D) Migration assay: Human Schwann cells were assessed for migration abilities on Boyden chambers assay for 4 hours with (A) various conditioned media +/−Slit2 antibody to deplete Slit2 from conditioned media, (B) Schwann cells conditioned media supplemented with 25 pg or 25 ng of Human recombinant Slit2, (C) conditioned media from control, mixed (F+M) or co-cultures (FcoM) using fibroblasts transfected with control or Slit2 targeting siRNA, (D) conditioned media from control, mixed (F+M) or co-cultures (FcoM) applied on Schwann cells transfected with control or Robo1 or Robo2 targeting siRNA. (A-D) (n=3) *, P<0.05; **, P<0.01; ***, P<0.001.

FIG. 2. Slit2 modulates N-cadherin/β-catenin signaling to influence Schwann cells migration ability. (A) N-cadherin/β-catenin binding was analyzed by coimmunoprecipitation in SNF cells after incubation with F+M or FcoM media. (n=3) **, P<0.01. (B) N-cadherin/β-catenin binding was analyzed by coimmunoprecipitation in SNF cells after incubation with F+M or FcoM media from fibroblasts transfected with Ctr (Si-Ctr) or Slit2 (Si-Slit2) siRNA. (n=3) *, P<0.05. (C) Nuclear extracts from SNF cells incubated with SNF, F+M or FcoM media were analyzed for β-catenin. Lamin A/C was used as control. β-catenin expression was corrected based on the level of Lamin A/C. (n=3) **, P<0.01. (D) mRNA level of 3 β-catenin targets (C-MYC, TCF4 and MMP9) analyzed by QRT-PCR in SNF cells after incubation with SNF, F+M or FcoM media. (n=3) **, P<0.01; ***, P<0.001.

FIG. 3. Slit2 influences PDA associated neural remodeling within in vivo mice models. (A) Correlation between αSMA expression and Slit2 expression in pancreatic tumor samples from 8 PDA bearing mice. The Pearson correlation test showed a positive and significant correlation of 0.919 (P<0.001). (B) Correlation between Slit2 score and number of intra-tumoral nerve (It-tum nerve) in pancreatic tumor samples from 12 PDA bearing mice. The Pearson correlation test showed a positive and significant correlation of 0.954 (P<0.001). (C) Correlation between Slit2 expression and number of intra-tumoral nerve (It-tum nerve) in 15 Human PDA xenograft samples. The Pearson correlation test showed a positive and significant correlation of 0.864 (P<0.001).

FIG. 4. SLIT2 is positively correlated with PDA associated neural remodeling in vivo. Count of nerve positive for Ki67 staining in Schwann cells, using 10 mice PDA samples with high (n=5) or low (n=5) SLIT2 level.

### EXAMPLE:

Material & Methods

Human Samples.

Freshly frozen tissue samples of PDAs (n=4) were obtained from patients who underwent surgery at the department of Digestive Surgery. Prior to surgery all patients had signed an informed consent form that had been approved by the local ethics committee; Agreement reference of CRO2 tissue collection: DC-2013-1857. One of the patients received preoperative chemotherapy during two months. Three patients underwent hemipancreaticoduodenectomy, and one distal pancreatectomy. No distant metastases were revealed at initial diagnosis. Histological examination confirmed diagnosis of PDA in all cases. Tumor staging was performed according to the International Union Against Cancer TNM System (the 6th edition).

Mouse Strains and Tissue Collection.

Pdx1-Cre;Ink4a/Arffl/fl;LSL-KrasG12D mice were obtained by crossing the following strains: Pdx1-Cre/KrasG12D/Ink4Af/f mice kindly provided by Dr. D. Melton (Harvard Stem Cell Institute, Cambridge, Mass.), Dr. R. Depinho (Dana-Farber Cancer Institute, Boston) and Dr. T Jacks (David H. Koch Institute for Integrative Cancer Research, Cambridge, Mass.), respectively. Pieces of tumor pancreata were fixed in 4% (wt/vol) formaldehyde for further immunostaining analysis or prepared for RNA extraction. All animal care and experimental procedures were performed in agreement with the Animal Ethics Committee of Marseille.

Xenografts.

Patient-derived pancreatic tumor pieces (1 mm3) were embedded in Matrigel before to be s.c. implanted into flank of adult male Swiss nude mice (Charles River Laboratories) under isoflurane anesthesia (induction, 4% (vol/vol); maintenance, 1.5% (vol/vol)). Experimental procedures related to the use of those patient-derived pancreatic tumor pieces were performed after agreement from the South Mediterranean Personal Protection Comity, under the reference 2011-A01439-32.

Laser Micro-Dissection and Microarray Analysis.

Microdissection was performed in the microdissection laboratory of the PRIMACEN plateform, University of Rouen, France, with the collaboration of Magalie Bénard. Frozen sections (20 μm) were obtained from selected tissue samples. After a brief staining with Hematoxylin and Eosin, sections were dehydrated. A surface of aproximatively 2.106 mm2 for epithelial compartment and 4.106 mm2 for stromal compartment has been microdissected, using the PALM system (P.A.L.M. Microlaser Technologies AG, Bernried, Germany). The microdissected material was immediately dissolute in a buffer containing β-mercaptoethanol and RNA carrier, and frozen before the RNA extraction was done with the RNAeasy Mini kit (Qiagen).

15 μg of total RNA was converted to cDNA by using Superscripts reverse transcriptase (Invitrogen), and T7-oligo-d(T)24 (Geneset) as a primer. Second-strand synthesis was performed using T4 DNA polymerase and E. Coli DNA ligase and then blunt ended by T4 polynucleotide kynase. cDNA was purified by phenol-chloroform extraction using phase lock gels (Brinkmann). Then cDNAs were in vitro transcribed for 16 h at 37° C. by using the IVT Labelling Kit (Affymetrix) to produce biotinylated cRNA. Labelled cRNA was isolated by using the RNeasy Mini Kit column (QIAGEN). Purified cRNA was fragmented to 200-30 mer using a fragmentation buffer. The quality of total RNA, cDNA synthesis, cRNA amplification and cRNA fragmentation was monitored by capillary electrophoresis (Bioanalizer 2100, Agilent Technologies). Fifteen micrograms of fragmented cRNA was hybridised for 16 h at 45° C. with constant rotation, using a human oligonucleotide array U133 Plus 2.0 (Genechip, Affymetrix, Santa Clara, Calif.). After hybridisation, chips were processed by using the Affymetrix GeneChip Fluidic Station 450 (protocol EukGE-WS2v5_450). Staining was made with streptavidin-conjugated phycoerythrin (SAPE, Molecular Probes), followed by amplification with a biotinylated anti-streptavidin antibody (Vector Laboratories), and by a second round of SAPE. Chips were scanned using a GeneChip Scanner 3000 G7 (Affymetrix) enabled for High-Resolution Scanning Images were extracted with the GeneChip Operating Software (Affymetrix GCOS v1.4). Quality control of microarray chips was performed using the AffyQCReport software.

The background subtraction and normalization of probe set intensities was performed using the method of Robust Multiarray Analysis (RMA) described by Irizarry et al. To identify differentially expressed genes, gene expression intensity was compared using a moderated t test and a Bayes smoothing approach developed for a low number of replicates. To correct for the effect of multiple testing, the false discovery rate, was estimated from p values derived from the moderated t test statistics. The analysis was performed using the affylmGUI Graphical User Interface for the limma microarray package, and Partek Genomics Suite (Partek Incorporated). We scored genes as differentially expressed if the fold-change was superior to 1.5 and p<0.05. Raw data were submitted to the GEO repository under the record number: GSE50570.

Cell Isolation and Primary CAFs Culture.

Small pancreatic tissue blocks were obtained during pancreas surgery from patients with resectable pancreatic adenocarcinoma (see Xenografts methods section). The tumor were cut into small pieces of 1 mm3 using a razor blade. The tissue pieces were digested by collagenase IV (Sigma C1889) for 30 minutes at 37° C., washed with media, resuspended, passed through cell strainer (100 uM) and finally plated in T75 cm2 flask. Tissue blocks trapped in cell strainer are seeded in 10 cm2 culture dishes in order to isolate more PSC by outgrowth. Cells were cultured in DMEM/F12 medium (Invitrogen, 31330-038), 10% serum (Sigma, F7524), 2 mmol/L L-glutamine (Invotrogen, 25030-024), 1% antibiotic/antimycotic (Invitrogen, 15240-062), 0.5% Sodium pyruvate (Invitrogen 11360-039) and used for passage 4 to 8. Primary CAFs features are verified by immunfluorescence for a positive □SMA staining and a negative CK19 staining.

In Vitro Modelling of Intra-Tumoral Microenvironment Cell Interactions.

Panc1 and MiaPaca 2 human cell lines; mouse pancreatic tumoral cell line PK4A, were used for epithelial compartment, Human primary fibroblasts or Human Cancer Associated Fibroblasts (CAFs) as well as murine macrophages (RAW 264.7) for stromal compartment, and Human Schwann cells (sNF 96.2) for nerve cell compartment. All cell lines were obtained from American Type Culture Collection, except PK4A, human fibroblasts and CAFs which are derived from primary cells lines obtained in our laboratory (see above methods paragraph). Cell lines were cultured in DMEM supplemented with 10% fetal bovine serum (Sigma, F7524) and 1% of antibiotic/antimycotic (Invitrogen, 15240-062). The combination of human and murine cell lines was important in our model as it permits to determine through QPCR analysis, by designing specific human or mouse primers, which gene expressions are modified in each cell type even when those cell types are co-cultured.

For modelling of intra-tumoral microenvironment cell interactions, fibroblasts and macrophage were cultured (cell concentration is dependent on dishes size) in dishes coated with collagen 0.1% (Sigma-Aldrich) alone or together (1:1) during 24 H and then serum deprived during 12 H. Panc1, MiaPaca 2 and sNF were cultured in uncoated dishes during 24 H and then serum deprived during 12 H. Conditioned medias (Md) from these cultures were used: Md F (Fibroblasts alone); Md M (Macrophages alone); Md FcoM (Fibroblasts co-cultured with Macrophages); Md F+M (Md from Fibroblasts alone mixed to Md from Macrophages alone; 1:1); Md SNF (sNF 96.2 alone).

Neuronexpert Assay.

Pregnant rats of 15 days gestation were killed by cervical dislocation (Wistar Rats, Janvier) and the fetuses were removed from the uterus. DRG were collected, placed in ice-cold Leibovitz medium (L15, Invitrogen) and dissociated by tripsinization (Trypsin, 0.05%, Invitrogen) for 20 min at 37° C. The reaction was stopped by addition of DMEM containing 10% of foetal bovine serum (FBS) in the presence of DNAase I (Roche). The suspension was triturated with a 10 mL pipette and cells will be then mechanically dissociated by several passages through the 21 gauge needle of a syringe. Cells were then centrifuged at 350×g for 10 min at room temperature. The pellet of dissociated cells was resuspended in DMEM-Ham F12 (Invitrogen) containing 1% N2 (invitrogen), 1% penicillin-streptomycin (Invitrogen), 1% L-glutamine and 3 ng/ml NGF (PeproTech and Tebu). Cells were seeded on the basis of 15000 cells per well in a 96-wells plate precoated with poly-L-Lysine (Sigma). Plates were maintained at 37° C. in a humidified incubator with 95% air/5% CO2. Cells were cultured in classic culture medium or in defined media culture. On day 5, cells were fixed by a solution of 4% paraformaldehyde in PBS at pH 7.4 for 30 min. After permeabilization with 0.01% saponin, cells were blocked for 2 h with PBS containing 10% goat serum, and then incubated with primary b-tubulin antibody (Sigma). Revelation is done using Alexa fluor 488 goat anti-mouse IgG (molecular Probe). Nuclei of neurons were labeled by a fluorescent marker (Hoechst solution, Sigma). For each condition, 2×10 pictures per well were taken using Analyzer™ 1000 (GE Healthcare) with 20× magnification. All images were taken in the same conditions and analysed with Developer Software (GE Healthcare).

Cell Migration Assay.

Schwann cell migration was studied using sNF96.2 cell line under various conditioned media on Boyden chambers. Culture inserts (BD Falcon) with a porous membrane at the bottom (8 g pores) were coated with a mix made of gelatin 0.1% and fibronectin 10 μg/ml, and then were seeded with sNF96.2 (100,000 per insert) and placed into wells containing the conditioned media. Migration was performed during 4 H. After cleaning and briefly staining inserts with coomassie blue, migration was assessed by counting the number of colored cells in 10 high power fields (Magnification 20×).

QRT-PCR.

RNA was extracted from cell lines using TRIzol (Invitrogen) according to the manufacturer's instructions. RNA from pancreas from 8-weeks old healthy mice (KrasG12D/Ink4AF/F) and PDA bearing mice (pdx1-cre/KrasG12D/Ink4AF/F) was extracted according to Chirgwin's procedure (64) and RNA quality control was determined using Agilent's 2100 Bioanalyzer. cDNA was made from 1 μg of total RNA using ImProm-II Reverse Transcription System (Promega) according to the manufacturer's instructions. QRT-PCR was performed using

cDNA amplicons amplified with specific primers and GoTaq qPCR Master Mix kit (Promega) using a Mx3000P Stratagene system. Relative expression was calculated as a ratio of the particular gene expression to a housekeeping gene expression (TBP).

Immuno Fluorescence.

Slides from frozen tissue samples or cultured cells were available for immunofluorescence. Staining was performed using Alpha smooth muscle actin (□SMA) mouse monoclonal (1:2, M-0851, DAKO or 1:200, A2547, Sigma-Aldrich), SLIT2 rabbit polyclonal (1:40, sc-28945, Santa Cruz Biotechnology), Cytokeratin 19 mouse monoclonal (1:50, M-0888, DAKO). Images quantification was done using Image J software.

Immunohistochemistry.

Slides from frozen human samples or formalin fixed mouse samples were available for immunohistochemistry. Staining was performed using SLIT2 rabbit polyclonal (1:40, sc-28945, Santa Cruz Biotechnology), PGP9.5 rabbit polyclonal (1:800, ab-10404, Abcam), AML mouse monoclonal (1:200, A2547, Sigma-Aldrich) antibodies.

Reagents.

Blocking SLIT2 antibody (rabbit polyclonal, 1 μg) was obtained from Santa Cruz Biotechnology (sc-28945). Human recombinant SLIT2 (25 pg or 25 ng) was obtained from Abcam (ab82131). Each was added to conditioned media during cell migration assays.

SiRNA Transfection.

Human fibroblasts were transiently transfected using SLIT2 siRNA (EHU068081, Sigma-Aldrich) or control siRNA (SIC001, Sigma-Aldrich) and ribocellin (BioCellChallenge) according to manufacturer's instructions. sNF96.2 cells were transiently transfected using ROBO1 and control siRNA (SR304090, Origene), ROBO2 and control siRNA (SR304091, Origene) and ribocellin (BioCellChallenge) according to manufacturer's instructions. Conditioned media produced by cells were serum deprived and then collected for migration assays, immunoprecipitation or cytoplasmic/nuclear protein extraction.

Immunoprecipitation.

sNF96.2 cells were incubated with conditioned media ±siRNA for 30 min. Cell layers were washed in cold PBS and incubated for 10 min in lysis buffer. Cell lysates were cleared by centrifugation at 15000 g for 15 min. Supernatant were incubated with N-cadherin antibody (rabbit polyclonal, 1 μg, ab18203, Abcam) for 2 H at 4° C. before addition of Agarose-beads. After 45 min of incubation with beads at 4° C., the material was washed three times with lysis buffer. The immunoprecipitated and input material was eluted in loading buffer, fractioned by SDS-PAGE, transferred to nitrocellulose membrane and immunoblotted with the appropriate antibody: N-cadherin (1:250, rabbit polyclonal, ab18203, Abcam), β-catenin (1:2000, mouse monoclonal, 610153, BD Transduction Laboratories).

Cytoplasmic and Nuclear Protein Extraction.

sNF96.2 cell were incubated with various conditioned media for 90 min. All steps were performed with Nuclear extract kit (Active Motif) according manufacturer's instructions. Nuclear extracts were resuspended in loading buffer, fractioned by SDS-PAGE, transferred to nitrocellulose membrane and immunoblotted with the appropriate antibodies: β-catenin (mouse monoclonal, BD Transduction Laboratories, 1:2000), Lamin A/C (rabbit polyclonal, Imgenex, 1/1000).

Western Blotting.

For detection of SLIT2, total proteins were isolated from human PDA and healthy pancreas. Proteins concentration of the lysates were determined by using the Bradford Protein Assay Reagent (Biorad). Electrophoresis was carried out using the XCell SureLock Mini-Cell (Invitrogen). The extracts (50 μg/lane) were resolved by 3-7% NuPAGE Novex Tris-Acetate Mini Gels electrophoresis and electrotransferred onto an Immobilon polyvinylidene difluoride (PVDF) membrane (Immobilon-PSQ) using an electrophoretic transfer system (Invitrogen). PVDF membranes were divided into two parts according to the location of molecular weight markers in order to permit detection of both C-terminal protein Slit2 (about 55-60 kDa) and β-tubulin (49 kDa) by Western blotting. The latter was used as an internal control. The membrane was blocked in freshly prepared PBS 1×, supplemented with 5% goat serum and 0.5% nonfat dry milk for 1 h at 37° C. The membrane was then incubated overnight at 4° C. in blocking buffer containing Slit2 (rabbit polyclonal antibody, 1:100, Santa Cruz Biotechnology) or β-tubulin antibody (mouse monoclonal antibody, 1:5000, Sigma) followed by three washes in TBST. Afterwards, the membrane was incubated with horseradish peroxidase-conjugated secondary antibody in TBS 1× supplemented with 3% BSA (1:5000 dilution, goat anti-rabbit or goat anti-mouse IgG-HRP, Santa Cruz Biotechnology) for 1 H at 37° C. The membranes were developed with an enhanced chemiluminescence substrate (Millipore), digitally scanned (Fusion Fx7 Vilber Lourmat).

Statistical Analysis.

Results are presented as average±standard deviation (SD). All other comparisons (except FIG. 6 and Supplemental FIG. 5) were analyzed by unpaired, two-sided, independent Student's test without equal variance assumption. Pearson correlation analysis (SAS Software 9.2) was run on comparisons between Slit2 and αSMA expression or nerve numbers.

Results

Determination of Stromal and Tumoral Cell Compartment Transcriptomic Signatures and Characterization of the Neurogenic Factor Family.

Clinical hallmarks of PDA are the abundant stroma reaction and the presence of PDA associated neural remodeling (data not shown), which have been widely separately documented. However, the connection between these two processes is limited to a unique study revealing that pancreatic cancer protein extracts induce neuronal plasticity. To analyze the connection between these two processes and how the intra-tumoral microenvironment impacts on neural remodeling, we decided to decipher the transcriptomic profile of stromal cell compartment within human PDA tissue, so called “PDA Stromal Signature”. We used laser capture microdissection (LCM) on human PDA samples to separate epithelial cells from stromal ones and analyzed their gene expression profiles using the Affimetrix U133 gene chip set (data not shown, GEO repository GSE50570). To understand how stromal compartment could impact on neural remodeling, we selected among genes significantly over-expressed within stromal compartment (1504 genes; P<0.05) those encoding for secreted or cell membrane proteins (S/CM), which represent a cluster of 753 genes that we named “PDA Stromal Secretome” (data not shown). Interestingly, after bio-informatic analyses through function-based databank software, we sorted out a sub-cluster of 122 genes involved in nervous system regulation. We decided to name this sub-cluster “Neurogenic factor family” which represents 16.2% of the “PDA Stromal Secretome” (data not shown). These data indicate that stromal compartment within PDA is specifically producing molecules that could impact on nerve cells abilities, and emphasizes our hypothesis that stromal compartment is physiologically impacting on PDA associated neural remodeling.

In Vitro Modeling of Intra-Tumoral Microenvironment Cell Interactions.

In order to functionally select the potential molecules that could be critically involved in PANR, from our original “Neurogenic Factor Family”, we optimized an in vitro model with heterotypic co-cultures. This model, constituted of human primary fibroblasts and murine macrophages (the two major cell components of PDA stromal compartment) cultivated on collagen matrix, mimics direct cell/cell connections (or communication through secreted molecules) occurring within intra-tumoral microenvironment (data not shown). The use of this model is perfectly relevant as we observed that primary fibroblasts co-cultivated with macrophages show increased expression of alpha-smooth muscle actin (αSMA), a well known Pancreatic Stellate cells (activated fibroblasts, PSCs or CAFs) marker within PDA (data not shown). This suggests that in co-culture condition with macrophages, autocrine/paracrine components are able to switch on the activation process of primary fibroblasts by turning them into stellate-like cells mimicking the intra-tumoral microenvironment. Our hypothesis, as well as previous data, suggests that the stromal compartment is able to secrete “neurogenic factors” potentially impacting on PANR. To verify this hypothesis, we analyzed the transcriptional expression of “neurogenic factor family” members in this in vitro heterotypic model (data not shown). As suspected, numerous “Neurogenic Factors” (i.e. BDNF, FYN, Neurotrimin, SerpinF1, Basp or Inhibin A) were specifically expressed by fibroblasts and/or macrophages or that their expression were enhanced in those cell type compare to 3 PDA epithelial tumoral cell lines (data not shown). Furthermore, we observed that some candidates from this family (i.e. FYN, Neurotrimin, Midkine, Cyr61 or SCHIP1) had their levels of expression increased in co-cultured fibroblasts with macrophages (data not shown). Compared to the significant modification of their expression in co-cultured fibroblasts with macrophages, only few candidates from the “Neurogenic Factor Family” showed an over-expression in co-cultured macrophages with fibroblasts (Neurotrimin, SerpinF1 and SCHIP1; data not shown), whatever suggesting that those co-cultured macrophages could have a modified phenotype, as it was recently reported. Those data suggest that our in vitro model can mimic the PDA intra-tumoral microenvironment as numerous candidates from stromal compartment were found enhanced in fibroblasts when they are co-cultured with macrophages. Therefore, our heterotypic co-culture could be used as an efficient in vitro model to decipher the modulation and the impact of intra-tumoral microenvironment on PDA progression, and in our case, on neural remodeling.

Media from Intra-Tumoral Microenvironment In Vitro Model Induce Changes in Neuron and Schwann Cell Behaviors.

So far, a unique study revealed that pancreatic tumor extracts (mix of proteins from every cell type composing PDA) could induce neuronal plasticity through increase of neurite density and neuronal branch length. To definitively confirm the strength of our in vitro model and also to emphasize our leading hypothesis on the role of stromal compartment on PANR, we first submitted dorsal root ganglion (DRG) neurons to our stromal conditioned media vs. control conditioned media. Interestingly, we observed that stromal conditioned media (FcoM) increases significantly the total number of neurons (relative fold change compare to control: 1.6±0.17, P<0.05), and more specifically large neurons (relative fold change compare to control of 1.5±0.21, P<0.05; data not shown), and favorize neuronal networks (data not shown) and branching pattern (relative fold change compare to control of 1.4±0.15 for 2 extensions, P<0.05, and 2.3±0.3 for 3 or more extensions, P<0.05; data not shown) which represent crucial parameters for nerve formation, extension and regeneration after an injury or within physiopathological circumstances. These data, while validating our cell co-culture model, on top of being correlated with previous observations, are further highlighting the direct implication of intra-tumoral microenvironment in neural remodeling associated processes.

However, while neuronal modulation is important for neural remodeling, processes associated with Schwann cells behavior (main nerve fibers cell components tightly associated with neurons) are also involved in fibers attraction and sprouting. Interestingly, we showed that stromal conditioned media (FcoM) enhances significantly Schwann cell proliferation, 5.75±0.4 vs. 3.6±0.3 for control condition (cell counting fold change; P<0.01; data not shown) and 1.0±0.05 vs. 0.6±0.08 for control condition (mitochondrial activity; P<0.05; data not shown) as well as Schwann cell migration with 14.3±2.7 for FcoM media vs. 5±2.5 for F+M media and 1±0.9 for SNF media (P<0.05 and P<0.01; data not shown). All together these data confirmed that intra-tumoral microenvironment is able to modify several neurons and Schwann cells abilities that can be related to processes involved in PDA associated neural remodeling.

Among Key Factors from Intra-Tumoral Microenvironment: Slit2, an Axon Guidance Molecule.

Regarding our data depicting neuron cells modified abilities (i.e. increased neuronal networks and extensions numbers) as well as our “neurogenic factor family” sub-cluster genes, we decided to pay specific attention on genes involved in the “Axon guidance” (data not shown). Indeed, as mentioned above, PDA associated neural remodeling is characterized by an increased nerve density in which axon guidance molecules could be involved in terms to impact on the attraction and growth of new and/or existing nerve fibers within PDA tumor. As already shown for some candidates (data not shown), we specifically analyzed the expression profile of the 14 genes present in the “axon guidance” cluster (data not shown). Among those 14 genes, we observed 4 members (Robo1, Robo2, Robo3 and Slit2) of a well known axon guidance family, the SLIT/ROBO signaling pathway which was recently associated with pancreatic cancer genome aberration and patient survival. Interestingly, Slit2, a gene coding for a secreted ligand known to activate the ROBO receptor and consequent pathway, is strongly expressed in fibroblasts compared to epithelial tumoral cell lines with a 120 fold increase (P<0.05; data not shown) and even more induced in fibroblasts co-cultured with macrophages with a fold increase of 1440 compared to its expression in tumoral cells (P<0.01; data not shown). This was confirmed at the protein level with an increased protein level of 5.1±0.3 from fibroblasts cultivated alone or co-cultured with macrophages (P<0.01; data not shown). These data suggest that, within PDA, SLIT2 should be expressed by PSCs, the activated fibroblasts. This was confirmed through in vivo localization in Human PDA where PSCs, expressing αSMA marker, are the main SLIT2 expressing cells (data not shown). As, the use of fibroblasts co-cultivated with macrophages was done as in vitro model in terms to mimic in vivo activated fibroblasts (PSCs), we verified the expression of Slit2 in primary PSCs isolated from Human PDA tissues. As expected, Slit2 expression was particularly enhanced in primary Human PSCs compared to epithelial tumoral cell with a fold increase range of 4.9e4 to 9.9e5 depending primary PSCs culture (P<0.001; data not shown). All together, those data reveal that intra-tumoral microenvironment of pancreatic adenocarcinoma, and more specifically Stellate cells, are able to produce several axon guidance related genes and among them, Slit2, suggesting a possible impact of the Slit2/Robo pathway on PDA associated neural remodeling and nerve density-associated changes.

SLIT2/ROBO Pathway Impacts on Neural Cells Behaviors Linked to PDA Associated Neural Remodeling.

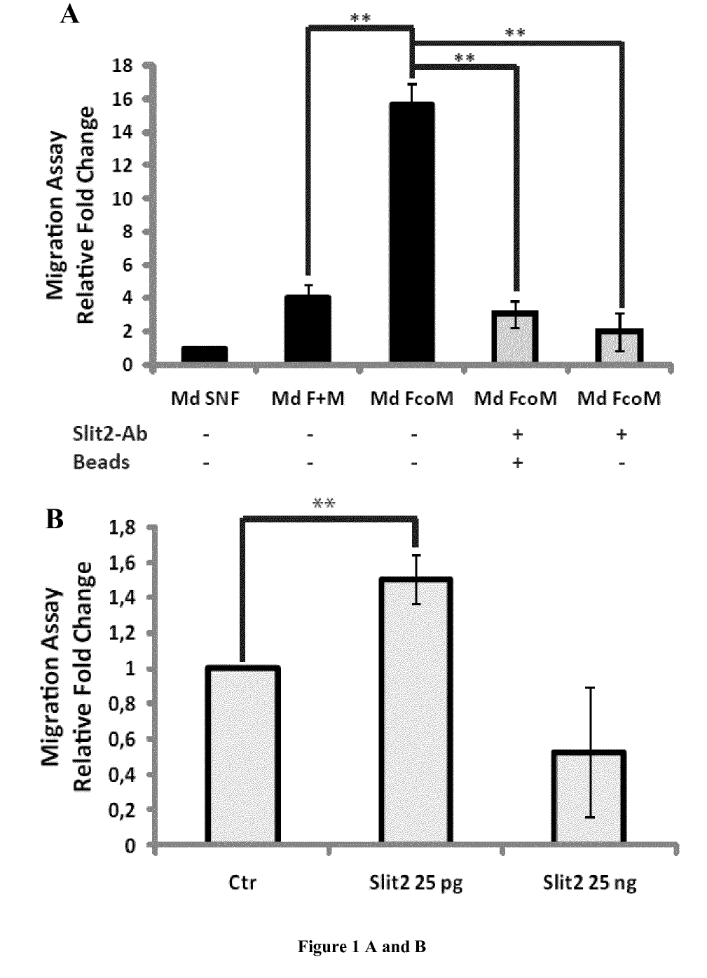

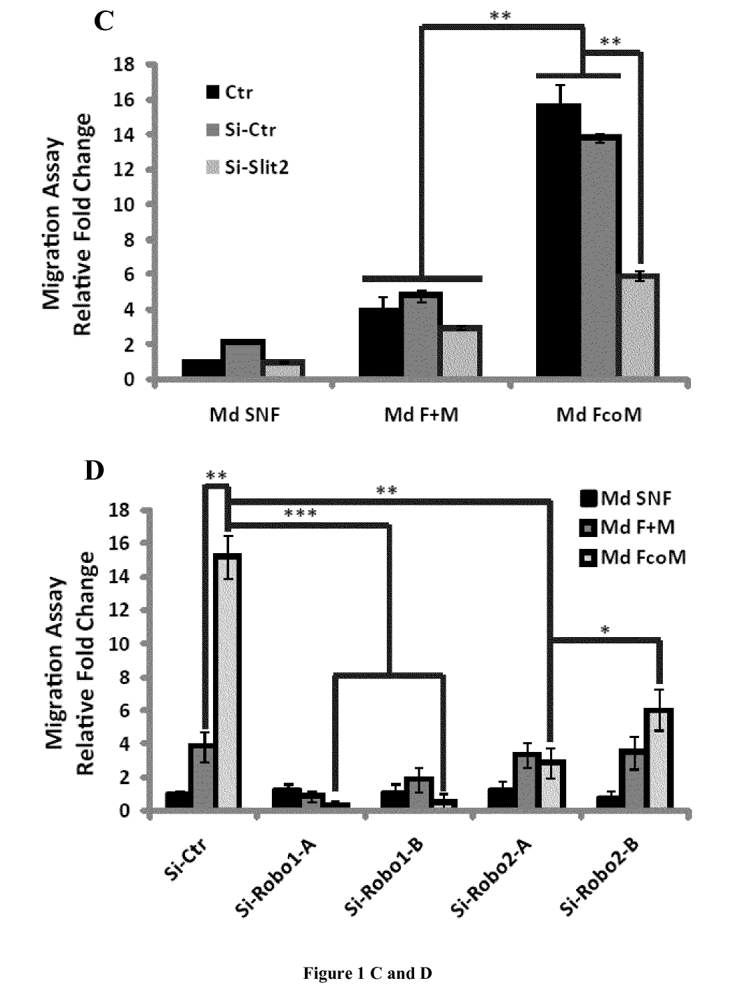

To determine the real impact of Slit2 and SLIT/ROBO signaling pathway on PANR, we took advantage of our in vitro model (data not shown). Following our hypothesis that Stellate cells are producing SLIT2 which is impacting on PANR, we first depleted secreted SLIT2 in conditioned medium. As suspected, we observed that the increase of Schwann cells migration induced by macrophages co-cultured fibroblasts conditioned medium (15.6 vs. 4.1 for F+M media, P<0.01) is lost after SLIT2-depletion (15.6 to 3.1 and 1.9, P<0.01; FIG. 1A). This means that SLIT2 within FcoM medium seems to be responsible of the enhanced Schwann cells migration. This was confirmed by using 25 pg of SLIT2 recombinant protein that enhanced Schwann cell migration of 1.5±0.1 fold (P<0.01; FIG. 1B). To further improve our hypothesis we silenced Slit2 mRNA in fibroblasts using siRNA in order to analyze the effects of F+M(-Slit2) or FcoM(-Slit2) conditioned media on Schwann cell migration. We showed that conditioned media from Slit2 deficient fibroblasts co-cultured with macrophages are no longer increasing Schwann cell migration (15.6 fold for FcoM Slit+ to 5.9 fold for FcoM Slit−, P<0.01; FIG. 1C). These data confirm that SLIT2 produced by fibroblasts when co-cultured/activated with macrophages is able to improve Schwann cell migration abilities.

Slit2 ligand is recognized and binds to members of the ROBO receptor family. To further strengthen the role of SLIT2/ROBO signaling in our study, we targeted Robo1 and Robo2 receptors mRNA in Schwann cells using siRNA strategies. Accordingly to previous experiments, we showed that Robo1 and Robo2 depletion in Schwann cells drastically impaired the induction of Schwann cell migration due to FcoM conditioned medium (P<0.001 for Robo1 depletion and P<0.05/0.01 for Robo2 depletion; FIG. 1D). All together those data strengthen our hypothesis on the impact of intra-tumoral microenvironment on Neural remodeling associated to pancreatic cancer through the implication of Slit2/Robo pathway.

Slit2 Modulates N-Cadherin/β-Catenin Signaling to Influence Schwann Cells Migratory Ability.

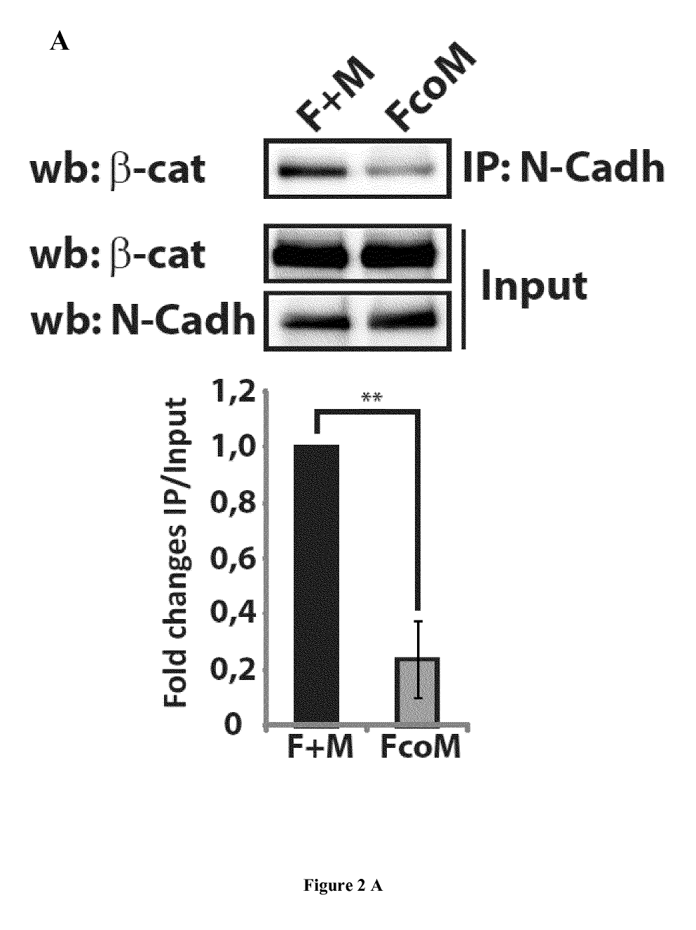

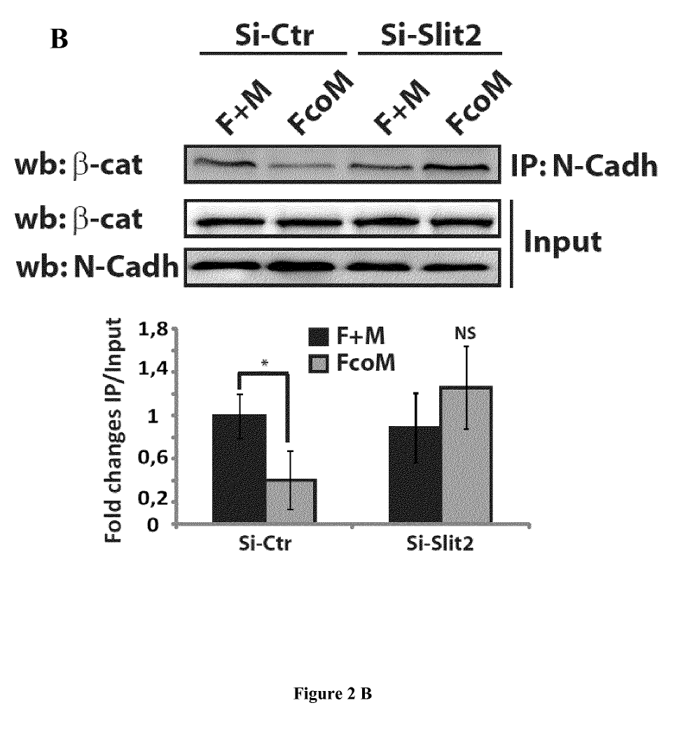

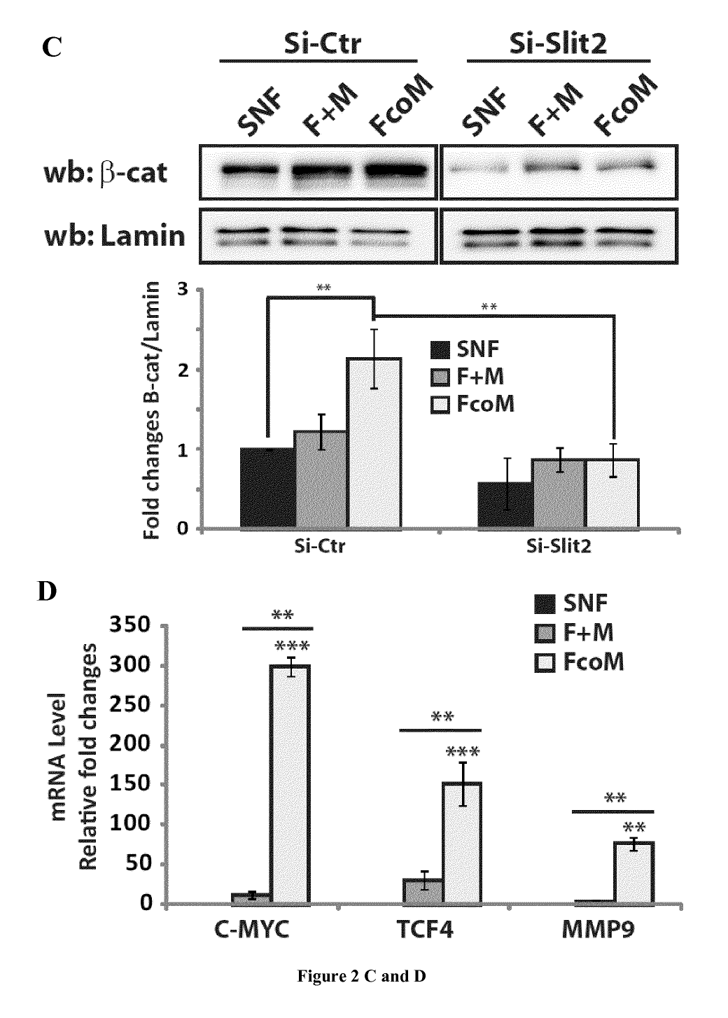

The highly conserved SLIT family, and their receptors ROBO, are well known to participate in central nervous system patterning as well as in sensory axon elongation and branching. Mechanistically, binding of Slit2 to Robo inhibits N-cadherin-mediated adhesion by inducing the separation of β-catenin from cytoplasmic part of N-cadherin. Moreover, it induces phosphorylation of β-catenin and its direct nuclear localization that alters transcription of migration/proliferation targets through TCF/LEF. Regarding those publications and the well established Slit2-activated pathways, we investigated if Slit2-mediated impact on Schwann cells abilities could be due to an activation of N-cadherin/β-catenin pathway in these cells. We first look at N-cadherin/β-catenin binding and observed that FcoM media decreases their co-immunoprecipitation, after 30 minutes (1 for F+M media vs. 0.2±0.1 for FcoM media, P<0.01; FIG. 2A). We confirmed that this effect was correlated to the presence of Slit2 within FcoM conditioned media by using conditioned media established with fibroblasts transfected with control (Si-Ctr) or Slit2 (Si-Slit2) siRNAs. Indeed, the use of FcoM media from si-Ctr treated fibroblasts co-cultured with macrophages showed a decrease in N-cadherin/β-catenin binding (1±0.2 vs. 0.4±0.3, P<0.05) while the use of FcoM media from si-Slit2 treated fibroblasts co-cultured with macrophages did not reveal any changes (0.9±0.3 vs. 1.3±0.3, NS; FIG. 2B). We then studied the consequent translocation of free β-catenin into the nucleus by analyzing nuclear extracts from SNF cells incubated with various conditioned media and revealed that FcoM media was increasing the nuclear β-catenin amount (1 vs. 2.2±0.3, P<0.01; FIG. 5C, left panel). This effect was related to presence of Slit2 as FcoM media from si-Slit2 treated fibroblasts co-cultured with macrophages could not induce such β-catenin translocation (2.2±0.3 vs. 0.9±0.2, P<0.01; FIG. 2C, right panel). Finally, we confirmed that nuclear β-catenin was transcriptionally active as FcoM media is able to significantly increase the expression level of some of its targets (C-MYC, TCF4 and MMP9) known to be related to Schwann cell migration (FIG. 2D). These results indicate that FcoM media through the presence of Slit2 is able to induce the activation of β-catenin pathway impacting on proliferative and migratory abilities of Schwann cells.

Slit2 Influences PDA Associated Neural Remodeling within In Vivo Models.

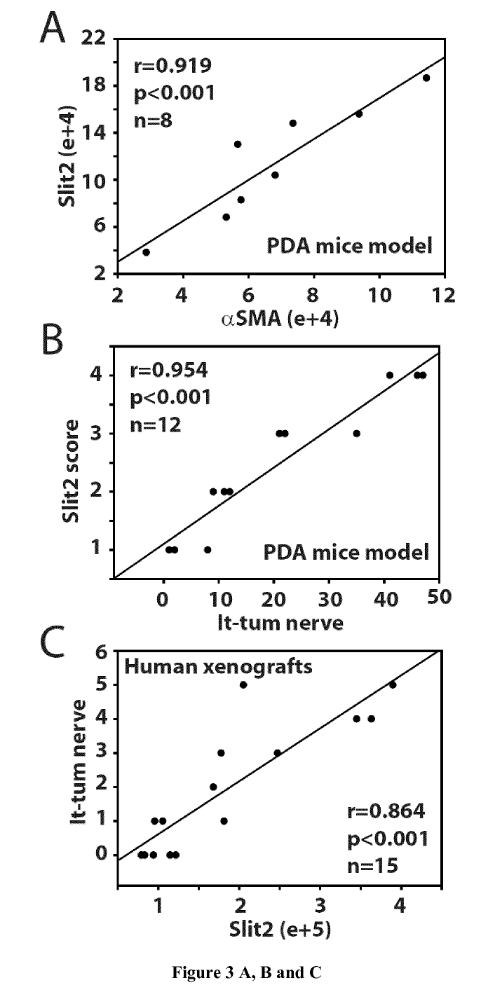

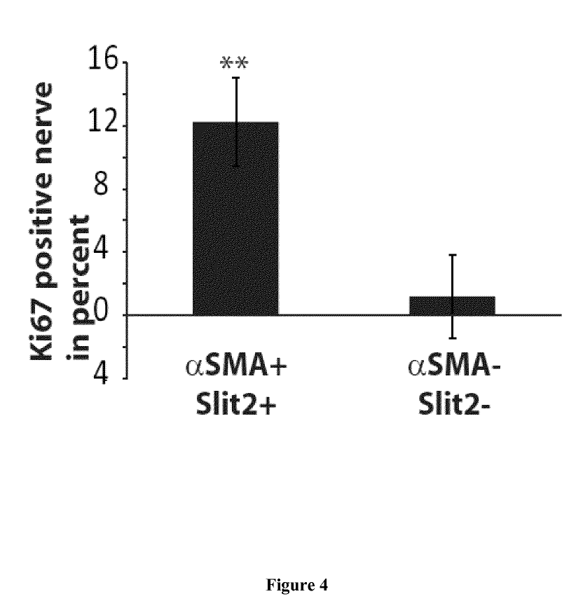

To investigate the relevance of Slit2 expression on PDA associated Neural Remodeling in vivo, we first determined if Slit2 was also present in PDA from an endogenous mice model; the pdx1-cre/KrasG12D/Ink4Af/f mice. As for Human PDA (data not shown), Slit2 expression was found increased in pancreatic tumor from pdx1-cre/KrasG12D/Ink4Af/f mice and more specifically in stromal compartment (data not shown). Moreover, we analyzed this stromal expression and revealed a significant correlation between the amount of αSMA and Slit2 staining in PDA from 8 different pdx1-cre/KrasG12D/Ink4Af/f mice (FIG. 3A and data not shown). This data reveals that Slit2 expression level is perfectly correlated with the amount of stromal compartment and PSCs within mice PDA. To determine if Slit2 expression level impacts on PDA associated Neural Remodeling and nerve density, we counted intra-tumoral and peri-tumoral nerves in PDA from 14 pdx1-cre/KrasG12D/Ink4Af/f mice, by using PGP9.5 IHC staining (data not shown). Interestingly, we found a positive and significant correlation (r=0.954; P<0.001), between intra-tumoral nerve and Slit2 expression (FIG. 3B). A positive correlation was also found between peri-tumoral as well as total nerve count and Slit2 expression (data not shown). To strengthen those in vivo data we decided to validate the correlation between Slit2 expression and intra-tumoral nerve density on human samples using xenograft tumors generated in nude mice by implanting pieces of freshly resected Human PDA tumors. In these xenograft tumor models, we also revealed a positive and significant correlation between nerve count (peri-, intra-, total) and SLIT2 level (P<0.001; FIG. 3C, data not shown). Finally, to correlate those in vivo data with previously shown information of SLIT2 impact on the proliferation rate of Schwann cells in vitro, we analyzed the proliferation rate of Schwann cells in vivo, in PDA tissues from endogenous mice models. We revealed that nerve with Ki67 positive Schwann cells are significantly increased in mice PDA tumors with high level of SLIT2 (αSMA+/SLIT2+) compare to nerve present in mice PDA with low level of SLIT2 (αSMA−/SLIT2−) (FIG. 4). All together, those data are consistent with our in vitro findings and confirm that SLIT2 expressed within PDA stromal compartment in vivo increases nerve fibers density within PDA tumors.

Conclusion:

Results presented in this patent application show clearly a real interest for the patients with PDA. In a clinical point of view, blockage of the SLIT2/ROBO pathway may be a relevant adjuvant therapeutic approach to reduce PDA associated Neural remodeling as well as consequent patho-physiologic impacts on PDA development and patient's fate as tumor recurrence and neuropathic pain. Use of compound of the invention could be of great benefit for overall survival through 2 processes; reduction of tumor recurrence and metastasis but also improvement of patient life quality through decrease of neuropathic pain. It's important to note that reduction of neuropathic pain and improvement of general well being of the patient could lead to maintained or even increased dose in chemotherapeutic protocols, which are often slow down or decreased due to overall decreased life quality.

### REFERENCES

Throughout this application, various references describe the state of the art to which this invention pertains. The disclosures of these references are hereby incorporated by reference into the present disclosure.

Ceyhan G O, et al. Pancreatic neuropathy results in “neural remodeling” and altered pancreatic innervations in chronic pancreatitis and pancreatic cancer. Am J Gastroenterol. 2009; 104(10):2555-2565.

Deshmukh S D, Willmann J K, Jeffrey R B. Pathway of extrapancreatic perineural invasion by pancreatic adenocarcinoma: evaluation with 3D volume-rendered MDCT imaging. Am. J. Roentgenol. 2010; 194:668-674.

Dickinson R E, Fegan K S, Ren X, Hillier S G, Duncan W C. Glucocorticoid regulation of SLIT/ROBO tumour suppressor genes in the ovarian surface epithelium and ovarian cancer cells. PLoS One. 2011; 6(11):e27792. doi: 10.1371/journal.pone.0027792. Epub 2011 Nov. 23.

Hanahan D, Weinberg R A. Hallmarks of cancer: The next generation. Cell. 2011; 144:646-674.

Hibi T, et al. Synuclein-gamma is closely involved in perineural invasion and distant metastasis in mouse models and is a novel prognosis factor in pancreatic cancer. Clin Cancer Res. 2009; 15(8):2864-2871.

Huang L, Xu Y, Yu W, Li X, Liqun C, He X, Peiying H. Robo1: a potential role in ocular angiogenesis. Curr Eye Res. 2009 December; 34(12):1019-29. doi: 10.3109/02713680903308495.

Hivert B, Liu Z, Chuang C Y, Doherty P, Sundaresan V. Robo1 and Robo2 are homophilic binding molecules that promote axonal growth. Mol Cell Neurosci. 2002 December; 21(4):534-45.

Kayahara M, Nakagawara H, Kitagawa H, Ohta T. The nature of neural invasion by pancreatic cancer. Pancreas. 2007; 35(3):218-223.

Lindsay T H, et al. Pancreatic cancer pain and its correlation with changes in tumor vasculature, macrophage infiltration, neuronal innervations, body weight and disease progression. Pain. 2005; 119(1-3):233-246.

Mahadevan D, Von Hoff D D. Tumor-stroma interactions in pancreatic ductal adenocarcinoma. Mol Cancer Ther. 2007; 6(4):1186-1197.

Ozaki H, et al. The prognostic significance of lymph node metastasis and intrapancreatic perineural invasion in pancreatic cancer after curative resection. Surg today. 1999; 29(1): 16-22.

Shimada K, et al. Intrapancreatic nerve invasion as a predictor for recurrence after pancreaticoduodenectomy in patients with invasive ductal carcinoma of the pancreas. Pancreas. 2011; 40(3):464-468.

Wang L J, Zhao Y, Han B, Ma Y G, Zhang J, Yang D M, Mao J W, Tang F T, Li W D, Yang Y, Wang R, Geng J G. Targeting Slit-Roundabout signaling inhibits tumor angiogenesis in chemical-induced squamous cell carcinogenesis. Cancer Sci. 2008 March; 99(3):510-7. doi: 10.1111/j.1349-7006.2007.00721.x. Epub 2008 Jan. 14.

1-6. (canceled)

7. A method for inhibiting the neuronal remodelling in cancer treating neuropathic pain, slowing cancer progression and/or preventing metastasis in a subject with cancer, comprising
administering to the subject a therapeutically effective amount of a compound which inhibits the binding of SLIT2 to ROBO1 or ROBO2 or a compound which is an inhibitor of SLIT2, ROBO1 or ROBO2 gene expression, wherein the therapeutically effective amount is sufficient to inhibit neuronal remodelling, treat neuropathic pain, slow cancer progression and/or prevent metastasis.

8-9. (canceled)

10. The method of claim 7 wherein the cancer is a pancreatic cancer.

11. The method of claim 10, wherein the pancreatic cancer is pancreatic ductal adenocarcinoma.

12. A method for preventing cancer recurrence in a subject that previously had cancer, comprising
administering to the subject that previously had cancer a therapeutically effective amount of a compound which inhibits the binding of SLIT2 to ROBO1 or ROBO2 or a compound which is an inhibitor of SLIT2, ROBO1 or ROBO2 gene expression, wherein the therapeutically effective amount is sufficient to prevent cancer recurrence.
